(19) United States
(12) Patent Application Publication
(10) Pub. No.: US 2013/0231576 A1
(43) Pub. Date: Sep. 5, 2013

(54) SOMATIC DATA-MEASURING APPARATUS AND SOMATIC DATA MEASUREMENT METHOD

(71) Applicants: Hiroaki Tanaka, Fukuoka (JP); Takuro Matsuda, Fukuoka (JP)

(72) Inventors: Hiroaki Tanaka, Fukuoka (JP); Takuro Matsuda, Fukuoka (JP)

(73) Assignee: Fukuoka University, Fukuoka (JP)

(21) Appl. No.: 13/879,135

(22) PCT Filed: Oct. 11, 2011
(86) PCT No.: PCT/JP2011/073338

§ 371 Date: May 7, 2013

(30) Foreign Application Priority Data
Oct. 12, 2010 (JP) 2010-229873

(51) Int. Cl. A61B 5/0205

(52) U.S. Cl. CPC A61B 5/0205
USPC 600/484; 600/483; 600/513; 600/528

(57) ABSTRACT

Provided are a somatic data-measuring apparatus that can easily and accurately measure the optimal exercise intensity for the subject being measured, and a somatic data measurement method. The somatic data-measuring apparatus is provided with a heart sound-acquiring means that detects the subject's heart sounds and outputs same as heart sound data, a first heart sound-extracting means that detects the first heart sound on the basis of the heart sound data, a first heart sound amplitude-measuring means that measures the amplitude from the detected first heart sound and outputs same as first heart sound amplitude data, a heart rate-counting means that measures the subject's heart rate and outputs same as heart rate data, and an exercise intensity-computing means that computes the double product of the heart rate data and the first heart sound amplitude data as double product data and detects, as the optimal exercise intensity, the exercise intensity at which the approximation line, which approximates said double product data distribution, bends. Since the double product, which represents myocardial oxygen consumption, is effective as an index that accurately reflects the state of cardiac workload, it is possible to measure accurately the degree of workload on the heart.

### CROSS-REFERENCE TO RELATED APPLICATION

The present application is based on the International Application No. PCT/JP2011/073338 which was filed on Oct. 11, 2011 and claims priority under 35 U.S.C. §119 from Japanese Patent Application No. 2010-229873 which was filed on Oct. 12, 2010.

### BACKGROUND OF THE INVENTION

1. Field of the Invention

The invention relates to a technology for measuring various somatic data concerning heart, exercise intensity, etc.

2. Background Art

People, especially those having problems with obesity or metabolic syndrome, walk or jog in order to stay healthy. Furthermore, grade exercise is also useful for rehabilitation in patients of myocardinal infraction and hypertension.

In grade exercise, it is difficult to have advantages if load (intensity) is low, and on the contrary, there is a risk that too much load exerts a harmful influence on body. It is thus preferable to find optimal exercise intensity for each of the patients.

Japanese Patent Application Publications Nos. 2006-116161 and 2007-14777 suggest a technique concerning optimal exercise intensity.

A method of determining an optimal exercise intensity, suggested in Japanese Patent Application Publication No. 2006-116161, includes steps to perform grade exercise in gradual levels, recording amplitude of first heart sound (S1 sound) generated when atrioventricular valves are closed for each of grade exercise levels, onto a heart sound diagram through a heart sound microphone attached to the patient's chest, and determining optimal exercise intensity which is at HSBP when the amplitude of heart sound suddenly rises.

A method of determining an optimal exercise intensity, suggested in Japanese Patent Application Publication No. 2007-14777, includes steps to detect how much the degree of amplitude of first heart sound varies compared to the degree in which an exercise load intensity varies, detect how much the degree (a rate) of a period in which a heart extends varies in one heart cycle concerned to a degree by which an exercise load intensity varies, and to determine, if a degree by which a rate of heart diastolic time to one heart cycle is equal to or greater than a first standard degree at a bending point at which an amplitude of first heart sound varies, the bending point as optimal exercise intensity.

In the above-mentioned Japanese Patent Application Publications Nos. 2006-116161 and 2007-14777, optimal exercise intensity is determined by the value of amplitude of first heart sound alone. However, the judgment made only with amplitude of first heart sound as an index for determining optimal exercise intensity lacks accuracy. If a patient suffers from a heart disease there is a risk that optimal exercise intensity determined by the value of amplitude of first heart sound alone will be excessive, and result in possible harmful influence on the patient.

It is known that myocaridnal oxygen consumption is correlated with a degree of exercise load, and thus, can act as an index representing the same. Myocardinal oxygen consumption is calculated as a double product, specifically, (heart rate)×(an internal pressure in a ventricle), in which an internal pressure in a ventricle is replaced with a systolic blood pressure which can be measured at upper arm since it is difficult to measure an internal pressure in a ventricle. Though it is better to evaluate myocardinal oxygen consumption by the double product than to evaluate it only by an amplitude of first heart sound, and it is preferable that myocardinal oxygen consumption is calculated as a triple product defined by (heart rate)×(contractility of heart muscle)×(tension force at a wall of a ventricle) in order to further enhance accuracy. It is known that contraction of heart muscle can be replaced with amplitude of first heart sound, and a tension force at a wall of a ventricle can be replaced with amplitude of second heart sound.

However, second heart sound has amplitude smaller than that of first heart sound, and can be disrupted by noises during exercise, which makes it difficult to accurately measure second heart sound. Accordingly, if myocardinal oxygen consumption could be accurately measured, it would be possible to measure optimal exercise intensity, apply optimal exercise remedy to a patient, improve a condition of a patient, and enhance exercise capacity of a patient.

### SUMMARY OF THE INVENTION

Thus, it is a target of the present invention to provide an apparatus for detecting somatic data and a method of doing it both of which make it possible to readily and accurately measure optimal exercise intensity of a target person.

The inventors conducted the experiments on a lot of target persons, and discovered a correlation between systolic blood pressure and amplitude of first heart sound. The present invention is based on the discovery.

An apparatus for detecting somatic data, in accordance with the present invention includes a first unit for sampling heart sound of a target person while the target person is exercising, a second unit for detecting first heart sound on the basis of the heart sound a third unit for measuring an amplitude of the first heart sound a fourth unit for counting a heart rate of the target person while the target person is exercising, a fifth unit for storing heart rate of the target person to be measured when the target person is resting, and an amplitude of first heart sound of the target person to be measured when the target person is resting, and a sixth unit for computing a double product of a ratio between the heart rate of the target person to be measured when the target person is resting and the heart rate to be measured when the target person exercises, and a ratio between the amplitude of first heart sound of the target person to be measured when the target person is resting and the amplitude of first heart sound of the target person to be measured when the target person exercises, and detecting an optimal exercise intensity of the target person on the basis of the double product.

A method of detecting somatic data, in accordance with the present invention, includes first step of sampling heart sound of a target person to be measured while the target person is exercising, second step of detecting first heart sound on the basis of the heart sound, third step of measuring an amplitude of the first heart sound, fourth step of counting a heart rate of the target person to be measured while the target person is exercising, fifth step of storing heart rate of the target person to be measured when the target person is resting, and an amplitude of first heart sound of the target person to be measured when the target person is resting, and sixth step of computing a ratio between the heart rate of the target person to be measured when the target person is resting and the heart rate to be measured when the target person exercises, and a ratio between the amplitude of first heart sound of the target person to be measured when the target person is resting and the amplitude of first heart sound of the target person to be measured when the target person exercises, and detecting optimal exercise intensity of the target person on the basis of the double product.

In accordance with the present invention, the first unit first samples heart sound of a target person, and outputs the sampled heart sound as heart sound data. The second unit detects first heart sound on the basis of the heart sound data, and the third unit measures an amplitude on the basis of the first heart sound, and outputs the same as first heart sound amplitude data. Then, the fourth unit counts a heart rate of the target person, and outputs the same as heart rate data. Then, the sixth unit computes, as double product data, a double product of the heart rate data to the first heart sound amplitude data, and detects an optimal exercise intensity on the basis of the double product. Since a double product of an amplitude of first heart sound indicative of myocardinal oxygen consumption to a heart rate is more effective as an index accurately reflecting a condition of a load exerted on a heart than an index of amplitude of first heart sound alone, even if the target person had heart disease, it is possible to accurately measure a degree of load exerting on a patient's heart.

The sixth unit detects an exercise intensity at which a line approximate to a distribution of said double product is bending, as optimal exercise intensity, for instance.

The sixth unit may be designed to divide the double product into a first group covering the double product before bending point appears and a second group covering the double product after bending point appeared compute a regression line of the first group as a first approximation line, computes a regression line of the second group as a second approximation line, select, among combinations of the first approximation line and the second approximation line, a combination which minimize a sum of a residual sum of squares of the first approximation line and a residual sum of squares of the second approximation line, and detect an intersection point of the first and second approximation lines of the selected combination as the optimal exercise intensity, ensuring it is possible to obtain an optimal exercise intensity with high accuracy.

It is preferable that the apparatus includes also a seventh unit which puts the optimal exercise intensity detected by the sixth unit into a relational expression derived from a correlation between the optimal exercise intensity and a maximum volume of oxygen taken by a target person during grade exercise, obtained by measuring a plurality of target persons, to thereby detect a maximum volume of oxygen taken by the target person during grade exercise.

Since the seventh unit makes it possible to detect a maximum volume of oxygen taken by the target person while he/she is exercising, on the basis of optimal exercise intensity detected by the sixth unit, and a relational expression derived from a correlation between the optimal exercise intensity and the maximum volume of oxygen, it is possible to compute aerobic exercise capacity of the target person. Since optimal exercise intensity can be calculated on the basis of a double product of an amplitude of first heart sound to a heart rate, and a maximum volume of oxygen taken by a target person can be calculated on the basis of the optimal exercise intensity, it is possible to specifically measure aerobic exercise capacity of the target person on the basis of the thus calculated maximum volume of oxygen.

The fifth unit preferably stores central blood pressure to be measured when the target person is resting, in which case, it is preferable that the apparatus further includes an eighth unit for computing central blood pressure while the target person is being tested on the basis of central blood pressure to be measured while the target person is resting, in accordance with a ratio between the amplitude of first heart sound of the target person to be measured while the target person is resting as standard data and received from the first heart sound amplitude measuring means, and the amplitude of first heart sound of the target person to be measured while the target person is being tested.

The eighth unit may be designed to estimate central blood pressure of the target person while the target person is being tested, in accordance with a relational expression between central blood pressure and amplitude of the first heart sound, the relational expression being defined for each of target persons on the basis of central blood pressure and amplitude of first heart sound of a target person both to be measured while the target person is resting, and-central blood pressure and amplitude of first heart sound of a target person both to be measured while the target person exercises. Thus, it is possible to compute central blood pressure in accordance with a relational expression corresponding to each of target persons, and hence, central blood pressure can be computed with accuracy. Herein, the phrase “the target person is exercising” indicates that he/she is performing exercise, namely, he/she is moving his/her body.

It is preferable that the second unit includes a second-A unit for measuring electrocardiogram of the target person, a second-B unit for detecting an R-wave out of the electrocardiogram a second-C unit for generating a gate signal, in accordance with timing at which the R-wave is generated, indicative of a certain period including first heart sound corresponding to the R-wave, and a second-D unit for detecting first heart sound on the basis of the heart sound taken while the gate signal is being generated.

The second-A unit measures electrocardiogram of the target person, and the second-B unit detects an R-wave. The second-C unit generates a gate signal, in accordance with a timing at which the R-wave is generated, indicative of a certain period including first heart sound corresponding to the R-wave, and hence, the second-D unit can detect first heart sound included the gate signal.

In the method in accordance with the present invention, it is preferable that the sixth step includes detecting an exercise intensity at which a line approximate to a distribution of the double product is bending, as optimal exercise intensity.

In the method in accordance with the present invention, it is preferable that the sixth step includes dividing the double product into a first group covering the double product before a bending point appears and a second group covering the double product after a bending point appeared, computing a regression line of the first group as a first approximation line, computing a regression line of the second group as a second approximation line, selecting, among combinations of the first approximation line and the second approximation line, a combination which minimizes a sum of a residual sum of squares of the first approximation line and a residual sum of squares of the second approximation line, and detecting an intersection point of the first and second approximation lines of the selected combination as the optimal exercise intensity.

It is preferable that the method in accordance with the present invention further includes seventh step of putting the optimal exercise intensity detected in the sixth step into a relational expression derived from a correlation between the optimal exercise intensity and a maximum volume of oxygen taken by a target person during grade exercise, obtained by measuring a plurality of target persons, to thereby detect a maximum volume of oxygen taken by the target person during grade exercise.

In the method in accordance with the present invention, it is preferable that central blood pressure is measured when the target person is resting and stored in the fifth step, in which case, the method further includes eighth step of computing a central blood pressure while the target person is being tested on the basis of a central blood pressure to be measured while the target person is resting in accordance with a ratio between the amplitude of first heart sound of the target person to be measured while the target person is resting as standard data and received from the first heart sound amplitude measuring means, and the amplitude of first heart sound of the target person to be measured while the target person is being tested.

In the method in accordance with the present invention, it is preferable that the eighth step includes estimating a central blood pressure of the target person while the target person is being tested, in accordance with a relational expression between a central blood pressure and an amplitude of the first heart sound, the relational expression being defined for each of target persons on the basis of a central blood pressure and an amplitude of first heart sound of a target person both to be measured while the target person is resting, and a central blood pressure and an amplitude of first heart sound of a target person both to be measured while the target person exercises.

In the method in accordance with the present invention, it is preferable that the second step includes measuring electrocardiogram of the target person, detecting an R-wave out of the electrocardiogram, generating a gate signal, in accordance with timing at which the R-wave is generated, indicative of a certain period including first heart sound corresponding to the R-wave, and detecting first heart sound on the basis of the heart sound taken while the gate signal is being generated.

The present invention further provides a computer-readable storage medium containing a set of instructions for causing a computer to carry out a method of detecting somatic data, the set of instructions comprising first instruction for detecting first heart sound on the basis of heart sound of a target person to be measured while the target person exercises, second instruction for measuring an amplitude of the first heart sound, third instruction for storing a heart rate of the target person to be measured when the target person is resting, and an amplitude of first heart sound of the target person to be measured when the target person is resting, and fourth instruction for computing a double product of a ratio between the heart rate of the target person to be measured when the target person is resting and the heart rate to be measured when the target person exercises, and a ratio between the amplitude of first heart sound of the target person to be measured when the target person is resting and the amplitude of first heart sound of the target person to be measured when the target person exercises, and detecting an optimal exercise intensity of the target person on the basis of the double product.

It is preferable that the sixth step includes detecting an exercise intensity at which a line approximate to a distribution of the double product is bending, as optimal exercise intensity.

It is preferable that the fourth instruction includes dividing the double product into a first group covering the double product before a bending point appears and a second group covering the double product after a bending point appeared, computing a regression line of the first group as a first approximation line, computing a regression line of the second group as a second approximation line, selecting, among combinations of the first approximation line and the second approximation line, a combination which minimize a sum of a residual sum of squares of the first approximation line and a residual sum of squares of the second approximation line, and detecting an intersection point of the first and second approximation lines of the selected combination as the optimal exercise intensity.

It is preferable that the instructions further includes fifth instruction for putting the optimal exercise intensity detected by the fourth instruction into a relational expression derived from a correlation between the optimal exercise intensity and a maximum volume of oxygen taken by a target person during grade exercise, obtained by measuring a plurality of target persons, to thereby detect a maximum volume of oxygen taken by the target person during grade exercise.

It is preferable that a central blood pressure is measured when the target person is resting and stored in the third instruction, in which case, the instructions may further include sixth instruction for computing a central blood pressure while the target person is being tested on the basis of central blood pressure to be measured while the target person is resting, in accordance with a ratio between the amplitude of first heart sound of the target person to be measured while the target person is resting as standard data and received from the first heart sound amplitude measuring means, and the amplitude of first heart sound of the target person to be measured while the target person is being tested.

It is preferable that the sixth instruction includes estimating a central blood pressure of the target person while the target person is being tested, in accordance with a relational expression between a central blood pressure and an amplitude of the first heart sound, the relational expression being defined for each of target persons on the basis of a central blood pressure and an amplitude of first heart sound of a target person both to be measured while the target person is resting, and a central blood pressure and an amplitude of first heart sound of a target person both to be measured while the target person exercises.

It is preferable that the first instruction includes measuring electrocardiogram of the target person, detecting an R-wave out of the electrocardiogram, generating a gate signal, in accordance with a timing at which the R-wave is generated, indicative of a certain period including first heart sound corresponding to the R-wave, and detecting first heart sound on the basis of the heart sound taken while the gate signal is being generated.

The advantages obtained by the aforementioned present invention will be described hereinbelow.

In accordance with the present invention, it is possible to easily detect optimal exercise intensity only by measuring amplitude of first heart sound and heart rate, with higher accuracy than when optimal exercise intensity computed by amplitude of first heart sound alone. Furthermore, the present invention makes it possible to easily compute myocardinal oxygen consumption as a double product of amplitude of first heart sound to a heart rate without using a triple product of a heart rate, contraction of heart muscle, and a tension force at the wall of a ventricle.

The above and other objects and advantageous features of the present invention will be made apparent from the following description made with reference to the accompanying drawings, in which reference characters designate the same or similar parts throughout the drawings.

### BRIEF DESCRIPTION OF THE DRAWINGS

FIG. 1 illustrates a target person whose heart stress is to be measured by the apparatus for detecting somatic data, in accordance with the first embodiment of the present invention.

FIG. 2 is a block diagram of the apparatus illustrated in FIG. 1.

FIG. 3A is a block diagram of an example of the heart sound sampling means, and FIG. 3B is a block diagram of an example of the electrocardiogram measuring means.

FIG. 4A indicates a position at which heart sound and electrocardiogram are measured, and FIG. 4B is a perspective view of a sensor unit.

FIG. 5 illustrates examples of an electrocardiogram and a heart sound diagram.

FIGS. 6A, 6B, 6C and 6D are graphs each indicating a relation between amplitude of first heart sound and central blood pressure in each of four target persons.

FIG. 7 is a graph indicating a relation between exercise intensity and a double product.

FIG. 8 is a graph indicating relations among exercise intensity, a double product, and secretion quantity of adrenalin.

FIG. 9 is a graph indicating a relation between a double product and a triple product.

FIG. 10 is a graph indicating a relation between optimal exercise intensity and maximum volume of oxygen taken by a target person.

FIG. 11 is a block diagram of the apparatus for detecting somatic data, in accordance with the second embodiment of the present invention.

### DESCRIPTION OF THE PREFERRED EMBODIMENTS

### First Embodiment

The apparatus for detecting somatic data, in accordance with the first embodiment of the present invention, is explained hereinbelow with reference to the drawings.

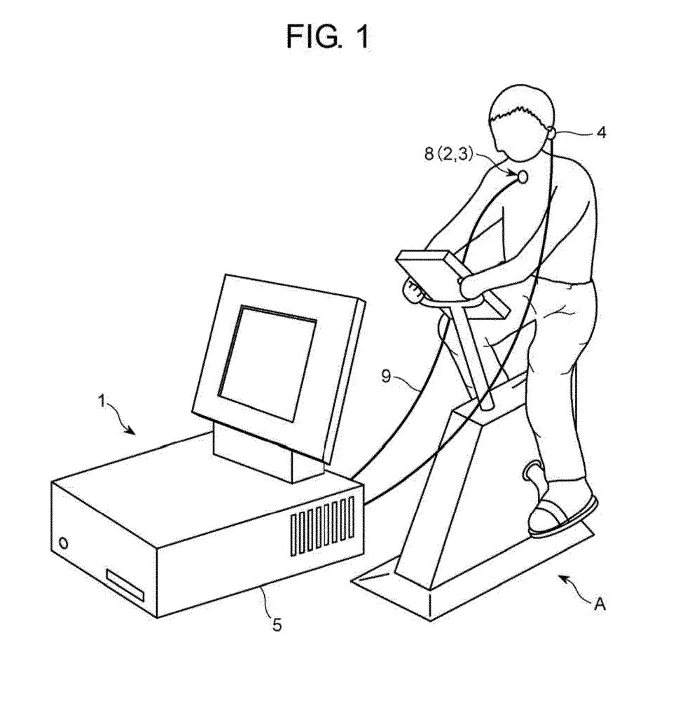

As illustrated in FIG. 1, the apparatus 1 for detecting somatic data measures a target person's condition when he/she is resting or when he/she is exercising through using exercise machine A to thereby provide various somatic data.

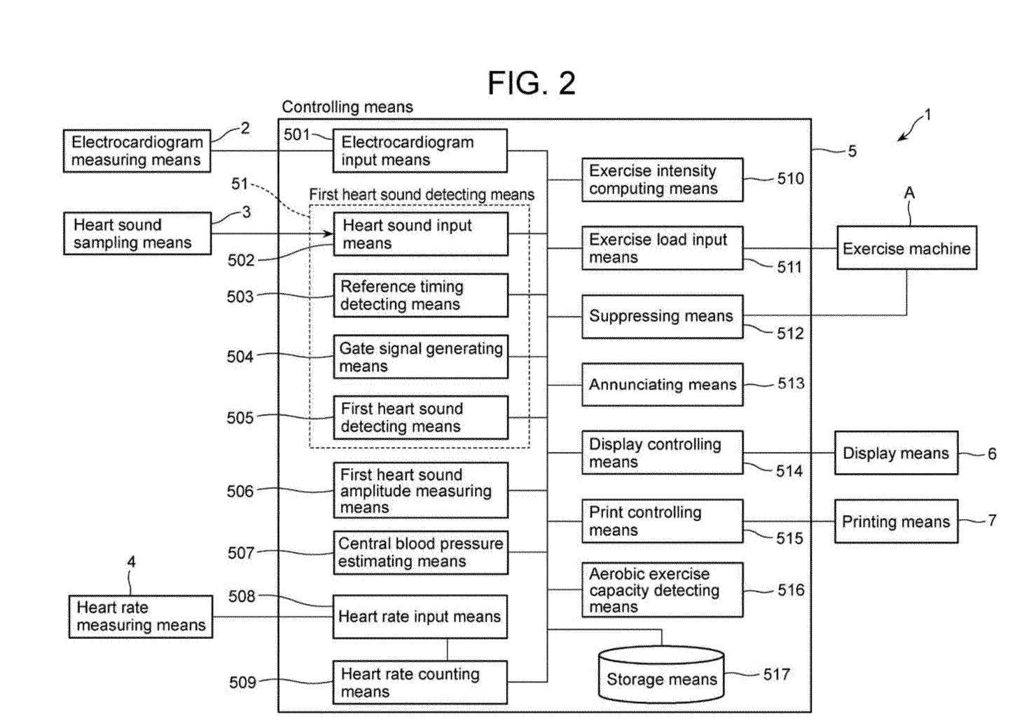

As illustrated in FIG. 2, the apparatus 1 includes an electrocardiogram measuring means 2, a heart sound sampling means 3, a heart rate measuring means 4, a controlling means 5, a display means 6, and a printing means 7.

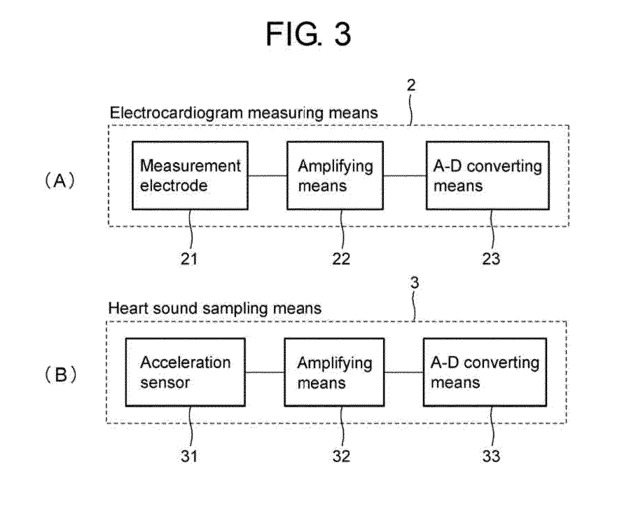

As illustrated in FIG. 3A, the electrocardiogram measuring means 2 may be designed to include a measurement electrode 21, an amplifying means 22, and an A-D converting means 23, for instance. The measurement electrode 21 includes two terminals for receiving an electric potential which generates on a target person's body when his/her heart pulsates, as electrocardiogram signals. The amplifying means 22 is comprised of an amplifier for amplifying the electrocardiogram signals. The A-D converting means 23 has a function of converting the amplified electrocardiogram signals into digital electrocardiogram data, and outputting the digital electrocardiogram data to the controlling means 5.

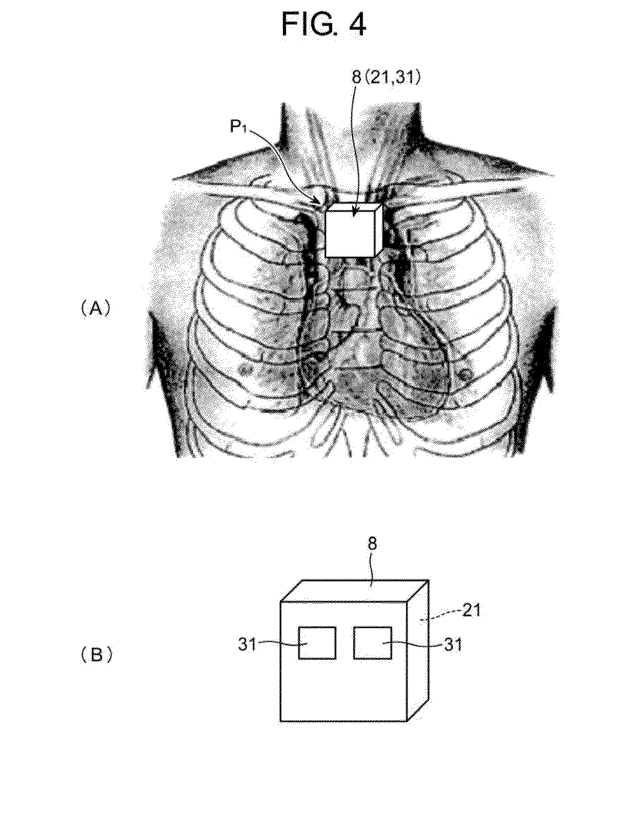

As illustrated in FIG. 3B, the heart sound sampling means 3 may be designed to include an acceleration sensor 31, an amplifying means 32, and an A-D converting means 33, for instance. In the first embodiment, the measurement electrode 21 and the acceleration sensor 31 are equipped together in a sensor unit 8 (see FIG. 4).

The acceleration sensor 31 senses acceleration in every movement direction, and has a function of sensing heart sound derived from pulsation of a target person's heart, as acceleration, and outputting the sensed acceleration as a heart sound signal. As illustrated in FIG. 4B, the acceleration sensor 31 is attached to a target person by means of an adhesive means such as a double-sided adhesive tape attached to a surface thereof. Any type of the acceleration sensor 31 can be employed, if it could sense acceleration in every direction.

For instance, the acceleration sensor 31 may be designed to be of an electrostatic capacity detection type in which acceleration is sensed by detecting a variation in a capacity between a movable part and a fixed part of a sensor element, a piezo-resistance type in which acceleration is sensed by detecting deformation of a spring caused by acceleration through the use of a piezo-resistance element equipped in the spring connecting a movable part with a fixed part of a sensor element, or a heat detection type in which acceleration is sensed by detecting, as a thermal resistance, a variation in convection of thermal air current generated by a heater, if the acceleration sensor 31 were designed to be of MEMS type. It is preferable that the acceleration sensor 31 is small-sized when attached to a target person in order not to disturb the target person while exercising, if the acceleration sensor 31 were designed to be any one of the above-mentioned types. The amplifying means 32 is comprised of an amplifier for amplifying a heart sound signal. The A-D converting means 33 has a function of converting the amplified heart sound signal into digital heart sound data, and outputting it to the controlling means 5.

As illustrated in FIG. 1, a lengthy single cable 9 extending to the controlling means 5 from the sensor unit 8 connects the electrocardiogram measuring means 2 to the connecting means 5, and further connects the heart sound sampling means 3 to the controlling means 5. Thus, a target person is not pulled by the cable 9 each time he/she exercises, to ensure he/she is not disturbed when he/she is performing grade exercise.

The heart rate measuring means 4 outputs a heart rate of a target person as heart rate data. The heart rate measuring means 4 may be designed to be able to be attached to a target person's earlobe, wrist or waist, or in the vicinity of a heart, for instance. In the first embodiment, the heart rate measuring means 4 is designed to be a clip type sandwiching an earlobe of a target person.

As illustrated in FIG. 2, the controlling means 5 measures a central blood pressure, a secretion volume of adrenalin, and a load exerted on the heart by computation, and may be comprised of a personal computer executing a program for measuring somatic data.

The controlling means 5 includes an electrocardiogram input means 501, a heart sound input means 502, a reference timing detecting means 503, a gate signal generating means 504, a first heart sound detecting means 505, a first heart sound amplitude measuring means 506, a central blood pressure estimating means 507, a heart rate input means 508, a heart rate counting means 509, an exercise intensity computing means 510, an exercise load input means 511, a suppressing means 512, an annunciating means 513, a display controlling means 514, a print controlling means 515, an aerobic exercise capacity detecting means 516, and a storage means 517.

The electrocardiogram input means 501 is comprised of an interface inputting the electrocardiogram data from the electrocardiogram measuring means 2 into the controlling means 5, and storing the same in the storage means 517. The heart sound input means 502 is comprised of an interface inputting heart sound data from the heart sound sampling means 3, and storing the same in the storage means 517.

The reference timing detecting means 503 has a function of detecting an R-wave on the basis of the electrocardiogram data stored in the storage means 517. The gate signal generating means 504 has a function of outputting a gate signal indicative of a certain period including first heart sound corresponding to the R-wave detected by the reference timing detecting means 503, that is, a period just prior to second heart sound. The first heart sound detecting means 505 has a function of detecting a peak waveform, as first heart sound, on the basis of the heart sound data made while the gate signal is being output. The first heart sound amplitude measuring means 506 measures an amplitude of the first heart sound detected by the first heart sound detecting means 505, and outputs the amplitude as first heart sound amplitude data.

The central blood pressure estimating means 507 has a function of making computation on the basis of the first heart sound amplitude data taken while a target person is being tested and received from the first heart sound amplitude measuring means 506, the first heart sound data (“resting” amplitude data) taken while a target person is resting and stored in the storage means 517, and central blood pressure data (“resting” central blood pressure data) indicative of a central blood pressure while a target person is resting, estimating a central blood pressure (a-systolic blood pressure) to be measured while a target person is being tested, and outputting the estimated central blood pressure as central blood pressure data.

The heart rate input means 508 is comprised of an interface inputting the heart rate data from the heart rate measuring means 4 into the controlling means 5, and storing the same in the storage means 517. The heart rate counting means 509 has a function of counting a heart rate on the basis of the heart rate data.

The exercise intensity computing means 510 has a function of multiplying the first heart sound amplitude data received from the first heart sound amplitude measuring means 506 with the heart rate data received from the heart rate counting means 509 to thereby calculate a double product as double product data (heart load data), and detecting a bending point at which a gradient bends relative to an exercise intensity, on the basis of the double product data.

The exercise load input means 511 is comprised of an interface inputting the exercise intensity data from the exercise machine A into the controlling means 5, and storing the exercise intensity data in the storage means 517.

The suppressing means 512 has a function of outputting a suppression signal to the exercise machine A in accordance with the optimal exercise intensity detected by the exercise intensity computing means 510 in order to prevent a grade exercise carried out by a target person from exceeding the optimal exercise intensity.

The annunciating means 513 has a function of making annunciation when the exercise intensity computing means 510 detects the bending point of the gradient or when exercise intensity exceeds a predetermined intensity beyond the bending point. Though the predetermined intensity is over the optimal exercise intensity, it is preferable that the predetermined intensity indicates such exercise load that is not an excessive load to a heart. The predetermined intensity can be determined for each target person. The annunciating means 513 may be designed to generate continuous or intermittent sounds or voice messages, turn on or flicker a lamp (not illustrated), or display messages on the display means 6 through the display controlling means 514.

The display controlling means 514 has a function of controlling display in the display means 6. The print controlling means 515 has a function of controlling printing carried out by the printing means 7.

The aerobic exercise capacity detecting means 516 has a function of detecting a maximum volume of oxygen taken by a target person during he/she is being loaded at maximum by exercise in accordance with both a relational expression (hereinbelow, the relational expression is called “aerobic exercise capacity calculation expression”) derived from a correlation between the optimal exercise intensity and a maximum volume of oxygen taken by a target person during grade exercise, the optimal exercise intensity detected by the exercise intensity computing means. The aerobic exercise capacity calculation expression is a linear function expressing a regression line indicative of a tendency of the distribution of a maximum volume of oxygen taken by a target person while he/she is exercising, and his/her optimal exercise intensity both obtained by sampling a plurality of target persons.

The storage means 517 is comprised of nonvolatile memory into which data can be written and from which data can be read. As the storage means 517, a hard disc device having a high capacity and enabling high-speed access can be employed. The storage means 517 stores therein heart sound data, electrocardiogram data and heart rate data when measured. Furthermore, the storage means 517 stores therein an amplitude of first heart sound, a central blood pressure, a heart rate, and a double product of an amplitude of first heart sound and a heart rate, all of which are measured when a target person is resting, as “resting” amplitude data, “resting” blood pressure data, “resting” heart rate data, and “resting” double product data.

In the first embodiment, the heart sound input means 502, the reference timing detecting means 503, the gate signal generating means 504, and the first heart sound detecting means 505 define the first heart sound detecting means 51.

The display means 6 may be designed to be comprised of CRT, LCD or organic EL display. The printing means 7 may be designed to be comprised of an ink-jet printer, a laser printer, a dot-impact printer or a thermal transfer printer all of which are capable of printing onto a paper.

The operation of the apparatus for detecting somatic data in accordance with the first embodiment of the present invention, having the above-mentioned structure, and a method of detecting somatic data are explained hereinbelow with reference to the drawings.

When a target person exercises, the sensor unit 8 including the acceleration sensor 31 and the measurement electrode 21 is attached to the target person. The acceleration sensor 31 is attached to a breast of the target person. The acceleration sensor 31 is preferably located above a sternum, as illustrated in FIG. 4A, and more preferably above a manubrium of sternum BP. The heart rate measuring means 4 is attached to an earlobe, wrist or waist, or in the vicinity of a heart.

Then, the target person starts exercising. In the exercise, the target person to which the sensor unit 8 is attached rides on the exercise machine A, specifically, a bicycle ergometer, and pedals continuously.

The heart sound signal transmitted from the acceleration sensor 31 is amplified by the amplifying means 32, and the A-D converting means 33 converts the heart sound signal having been amplified every certain period of time into heart sound data, namely, sampled digital data (heart sound detecting step). The electrocardiogram signal transmitted from the measurement electrode 21 is amplified by the amplifying means 22, and the A-D converting means 23 converts the electrocaridogram signal having been amplified every certain period of time into electrocardiogram data, namely, sampled digital data (electrocardiogram detecting step).

The heart sound input means 502 of the controlling means 5 receives the heart sound data from the heart sound sampling means 3, and stores the heart sound data into the storage means 517 together with the exercise intensity data received from the exercise machine A. The electrocardiogram input means 501 receives the electrocardiogram data from the electrocardiogram measuring means 2, and stores the electrocardiogram data into the storage means 517 together with the exercise intensity data received from the exercise machine A. The heart rate input means 508 receives the heart rate data from the heart rate measuring means 4, and stores the heart rate data into the storage means 517 (heart rate detecting step). The heart rate counting means 509 reads the heart rate data out of the storage means 517, calculates a heart rate, and stores the heart rate into the storage means 517 as heart rate data (heart rate counting step).

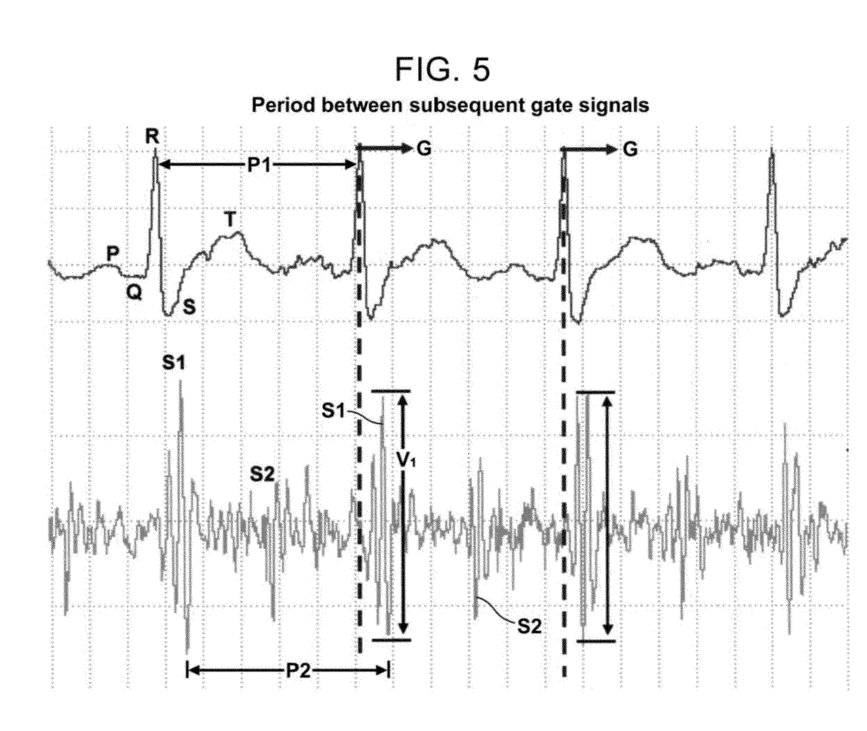

The reference timing detecting means 503 detects an R-wave on the basis of the electrocardiogram data stored in the storage means 517. Herein, an R-wave is explained hereinbelow with reference to FIG. 5.

Since an R-wave is observed at the end of heart's expansion, it is possible to use an R-wave as a reference for detecting both first heart sound generated when an auriculoventricular valve (a mitral valve, a tricuspid valve) is closed and second heart sound generated when an arterial valve (an aortic valve, a pulmonary valve) is closed while the heart is pulsating.

An R-wave has a higher peak than that of a P-wave, a Q-wave, a S-wave and a T-wave, and rises up more steeply than others. Accordingly, the reference timing detecting means 503 can relatively readily detect an R-wave by detecting electrocardiogram data having a highest peak.

On detection of an R-wave, the reference timing detecting means 503 transmits a signal indicating the detection of an R-wave, to the gate signal generating means 504 (reference timing detecting step).

On receipt of the signal indicating the detection of an R-wave, the gate signal generating means 504 generates a gate signal G indicative of a certain period including the first heart sound 51 corresponding to the R-wave, in accordance with a timing of the R-wave. The certain period may be determined as a period between a timing at which the R-wave is generated and the second heart sound S2. A period between the generation of an R-wave and the second heart sound S2 depends on individual, and further depends on exercise workload. Furthermore, a period between the generation of an R-wave and the first heart sound S1 depends on each of target persons' conditions. Accordingly, the certain period would not include the first heart sound S1, if the gate signal G were too short, and would include not only the first heart sound S1, but also the second heart sound S2, if the gate signal G were too long. Thus, a period determined by statistically measuring young to elderly persons is used as the certain period in the first embodiment.

The first heart sound detecting means 505 extracts a peak wave (the first heart sound S1) on the basis of the heart sound data taken while the gate signal G is being output (first heart sound detection step).

Heart sound extracted while the gate signal G is being output surely includes first heart sound S1. In other words, by limiting a range in which data corresponding to the first heart sound S1 is extracted among the heart sound data by value of the gate signal G, it is possible to remove the second heart sound S2 and noises. Immediately after a target person starts grade exercise, the first heart sound S1 is higher than the second heart sound S2 in some cases, in which it is not possible to discriminate the first and second sounds from each other only by amplitude. That is, it is difficult to identify the first heart sound only on the basis of the heart sound data. Consequently, in the apparatus 1 for detecting somatic data, in accordance with the first embodiment of the present invention, a gate signal as a reference is generated on the basis of an R-wave, and a peak waveform is detected as the first heart sound S1 in a period indicated by the gate signal, ensuring it possible to extract data of the first heart sound S1.

The first heart sound amplitude measuring means 506 measures an amplitude V1 of a peak waveform, and stores the amplitude V1 into the storage means 517 as first heart sound amplitude data (first heart sound amplitude measuring data).

The first heart sound amplitude measuring means 506 is able to read ten first heart sound amplitude data out of the storage means 517, calculate an average of them, and store the average as single first heart sound amplitude data into the storage means 517. By averaging a certain number of first heart sound amplitude data, even if the amplitudes of the first heart sound S1 had a fluctuation, it is possible to reduce its overall influence. Though ten first heart sound amplitude data is simply averaged in the first embodiment, a plurality of first heart sound amplitude data may be averaged by other statistical processes (first heart sound averaging step).

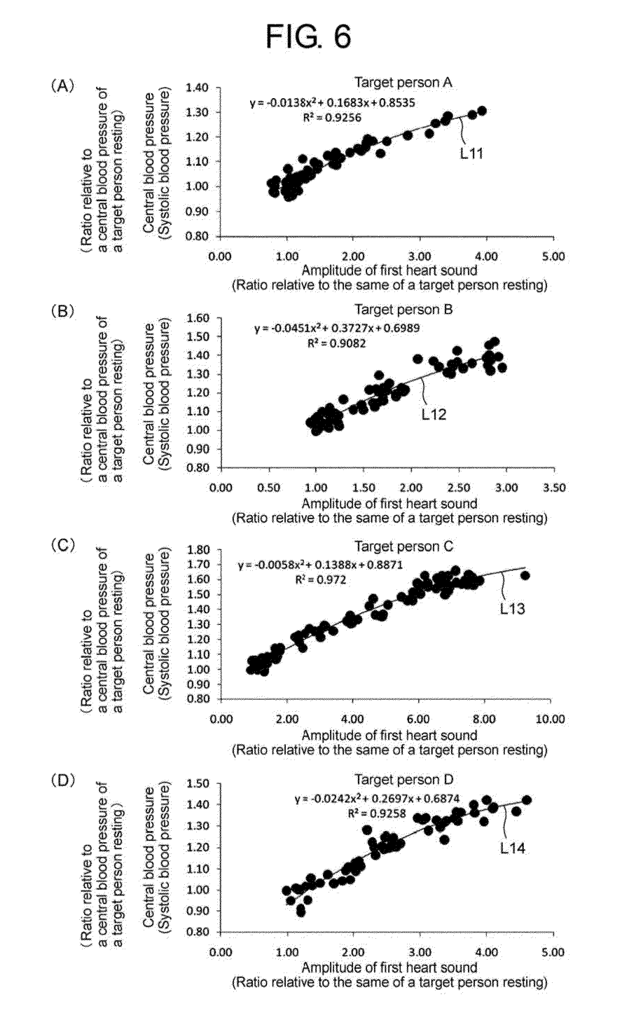

The central blood pressure estimating means 507 divides the first heart sound amplitude data measured by the first heart sound amplitude measuring means 506 when a target person is being tested, by the first heart sound amplitude data (amplitude data measured while a target person is resting) measured as reference data while a target person is resting and transferred to the storage means 517 from the first heart sound amplitude measuring means 506, to thereby have a ratio. The central blood pressure estimating means 507 further computes a central blood pressure of a target person being tested on the basis of the central blood pressure data measured while a target person is resting, read out of the storage means 517, in accordance with the ratio, and outputs it as central blood pressure data (central blood pressure estimating step). The computation is carried out on the basis of a relational expression between amplitude of first heart sound and a central blood pressure. Herein, the relational expression between amplitude of first heart sound and central blood pressure is explained hereinbelow with reference to FIG. 6.

The graphs illustrated in FIGS. 6A to 6D indicate an amplitude of first heart sound and central blood pressure (systolic blood pressure) of four target persons, having been measured with a grade exercise intensity gradually increased from resting condition to a condition in which a heavy load acts on them. The central blood pressure was measured by a conventional method, that is, by inserting a catheter through wrist and locating the catheter in the vicinity of a heart. The four target persons are all men in their twenties in good health. Each of the graphs has an axis of abscissas (x-axis) indicating a ratio between amplitude of first heart sound measured when a target person is resting and an amplitude of first heart sound measured when a target person exercises, and an axis of ordinates (y-axis) indicating a ratio between a central blood pressure (a systolic phase) measured when a target person is resting and a central blood pressure measured when a target person exercises.

Thus, it was found that amplitude of first heart sound has a correlation with a central blood pressure, as shown in the graphs illustrated in FIGS. 6A to 6D. The correlation can be expressed as the approximation straight line L11 to L14.

For instance, a relational expression indicating the approximation line L11 of a target person A can be expressed by the following expression (1).

y=−0.0138x²+0.1683x+0.8535 (1)

Estimating by Pearson's correlation coefficients, since the contribution rate R² was 0.9256, it was found that the relational expression (1) had a high correlation, and could be applied to almost all cases.

Accordingly, supposing that “x1” indicates the first heart sound amplitude data indicating an amplitude of first heart sound measured when a target person is resting, “x2” indicates the first heart sound amplitude data indicating an amplitude of first heart sound measured when a target person is being tested, “y1” indicates the central blood pressure data indicating central blood pressure measured when a target person is resting, and “y2” indicates the central blood pressure data indicating central blood pressure measured when a target person is being tested, the central blood pressure “y2” indicating central blood pressure measured when a target person is being tested can be expressed by the following equation (2).

y2=(−0.0138(x2/x1)²+0.1683(x2/x1)+0.8535)×y1 (2)

The central blood pressure estimating means 507 can accurately calculate central blood pressure to be measured when a target person is being tested (or, is exercising), by carrying out the calculation in accordance with the relational expression (2).

Similarly, the relational expressions (3) to (5) indicating the approximation straight line L12 to L14 of the target persons B to D are as follows.

y=−0.0451x²+0.3727x+0.6989 (3)

y=−0.0058x²+0.1388x+0.8871 (4)

y=−0.0242x²+0.2697x+0.6874 (5)

With respect to the target persons B to D, the contribution rates R² for the target persons B, C and D were 0.9082, 0.972 and 0.9258, respectively, indicating a high correlation. Accordingly, it is possible to estimate central blood pressure of the target persons B to D in accordance with the computation based on the relational expressions (3) to (5).

It is preferable that the computation used by the central blood pressure estimating means 507 for estimation of central blood pressure is determined for each of target persons. Specifically, an amplitude of first heart sound and central blood pressure (systolic blood pressure) are measured for each of target persons by a conventional process with a grade exercise intensity gradually increased from a resting condition to a condition in which heavy load acts on them, to thereby introduce a relational expression between central blood pressure and amplitude of first heart sound. Then, the relational expression is converted into an expression used for computing central blood pressure to be measured when a target person is being tested, and the obtained expression is stored in the storage means 517 in association with identification data (for instance, a name or an ID number) of each of target persons. When a target person exercises, the central blood pressure estimating means 507 reads the expression associated with a target person out of the storage means 517, and computes central blood pressure to be measured while a target person is being tested, ensuring that the measurement can be carried out with high accuracy even when a target person is exercising.

It was impossible in a conventional process to accurately measure central blood pressure while a target person is exercising, because the measurement was carried out by inserting a catheter through wrist and locating the catheter in the vicinity of a heart. In the apparatus for detecting somatic data in accordance with the first embodiment, it is possible to accurately and readily measure central blood pressure regardless of whether a target person is resting or exercising, by measuring an amplitude of first heart sound, and estimating central blood pressure to be measured when a target person is exercising, on the basis of the amplitude of first heart sound. Thus, it is possible to safely and effectively evaluate exercise and/or exercise remedy for the purpose of keeping healthy, by means of an inexpensive tool.

Even if the expression were not prepared for a certain target person, it would be possible to estimate his/her central blood pressure by using an expression prepared for other target person, since central blood pressure has a correlation with amplitude of first heart sound, though accuracy is slightly reduced.

Hereinbelow the calculation of a double product carried out by the exercise intensity computing means 510 is explained. When a request of calculating a double product is input through an input means (not illustrated), and is stored in the storage means 517, the exercise intensity computing means 510 computes a double product of heart rate data and first heart sound amplitude data both stored in the storage means 517, and outputs the computed double product as double product data (data indicative of a load acting on a heart).

In the first embodiment, the double product data is comprised of a double product data of a rate between a heart rate measured when a target person is resting and a heart rate measured when a target person is tested (exercises), and a rate between an amplitude of first heart sound measured when a target person is resting and an amplitude of first heart sound measured when a target person is tested.

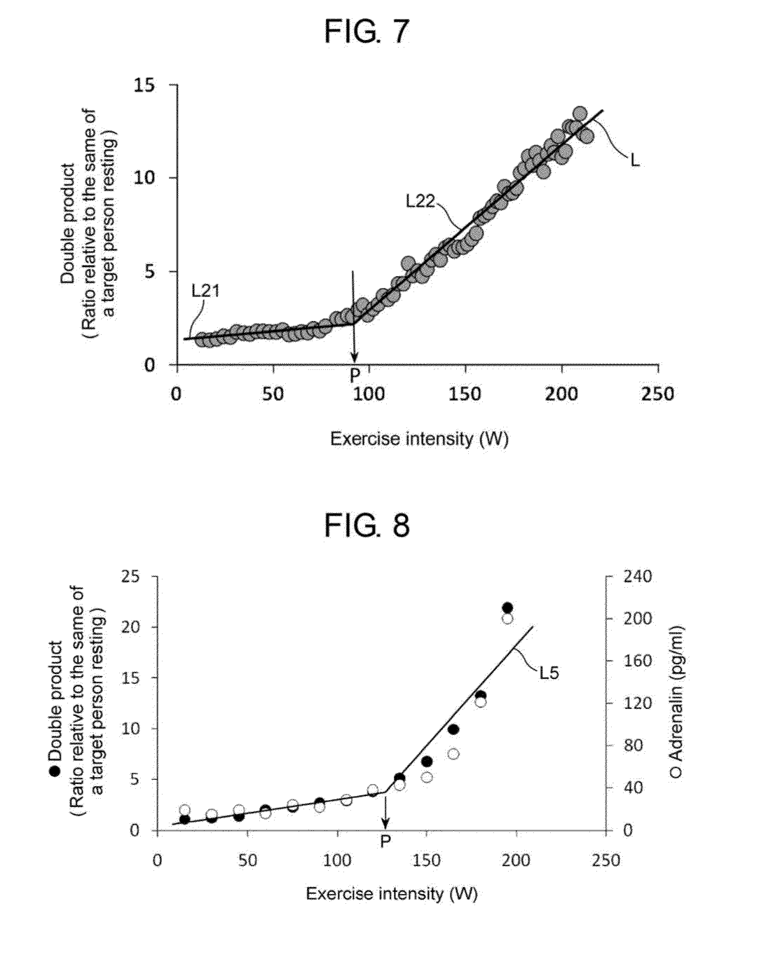

The exercise intensity (exercise intensity data) and the double product (data indicative of load acting on the heart) of the target person is shown in FIG. 6B, who is a man in his twenties in good health, are plotted in the graph illustrated in FIG. 7. The graph illustrated in FIG. 7 has an axis of ordinates (Y-axis) indicative of a double product, and an axis of abscissas (X-axis) indicative of exercise intensity. The data shown in the graph is obtained by the exercise intensity computing means 510, specifically, by reading the first heart sound amplitude data to be measured when a target person is resting and the heart rate data to be measured when a target person is resting, out of the storage means 517, computing a ratio between the first heart sound amplitude data to be measured when a target person is resting and the first heart sound amplitude data to be measured when a target person exercises, and further a ratio between the heart rate data to be measured when a target person is resting and the heart rate data to be measured when a target person exercises, and multiplying those ratios to thereby compute the double product data.

The graph indicates that the bending point P at which a gradient of the approximation line L suddenly increases is the optimal exercise intensity.

Herein, how the exercise intensity computing means 510 determines the bending point P is explained in detail.

Supposing that “x” indicates an exercise intensity and “y” indicates a double product, it is supposed that there are obtained n data (x₁, y₁), (x₂, y₂) , , , (xₙ, yₙ). Supposing that a line suitable as a regression line can be expressed as “y=ax+b”, “a” and “b” can be calculated in accordance with the following expressions (6) and (7).

$$\begin{matrix}a = \frac{n {\sum }_{k = 1}^{n} {x}_{k} {y}_{k} - {\sum }_{k = 1}^{n} {x}_{k} {\sum }_{k = 1}^{n} {y}_{k}}{n {\sum }_{k = 1}^{n} {x}_{k}^{2} - {( {\sum }_{k = 1}^{n} {x}_{k} )}^{2}} & ( 6 ) \\ b = \frac{{\sum }_{k = 1}^{n} {x}_{k}^{2} {\sum }_{k = 1}^{n} {y}_{k} - {\sum }_{k = 1}^{n} {x}_{k} {y}_{k} {\sum }_{k = 1}^{n} {x}_{k}}{n {\sum }_{k = 1}^{n} {x}_{k}^{2} - {( {\sum }_{k = 1}^{n} {x}_{k} )}^{2}} & ( 7 )\end{matrix}$$

In the judgement conducted by the exercise intensity computing means 510 for an optimal exercise intensity, since the graph of the double product in which exercise load gradually increases indicates an exponential curve, the data is divided into two groups at a boundary of appearance of the bending point P, specifically, first group covering the data obtained before the bending point P appears, and second group covering the data after the bending point P appeared.

Then, the exercise intensity computing means 510 computes regression lines as first and second approximation straight lines in accordance with the above-mentioned expressions (1) and (2) on the basis of both the first group data and the second group data. Then, the exercise intensity computing means 510 selects, among a lot of combinations of the first and second approximation straight lines, a combination of the first and second approximation straight lines which minimizes a residual sum of squares of the first and second approximation straight lines. Then, the exercise intensity computing means 510 judges “x” of the intersection (the bending point P) of the selected first approximation straight line L21 with the selected second approximation straight line L22, as optimal exercise intensity.

In the way as mentioned above, the exercise intensity computing means 510 computes the approximation line L comprised of polygonal lines, on the basis of data indicative a load acting on a heart, that is, data of a double product obtained by multiplying an amplitude of first heart sound indicative of a cardiac contractile force with a heart rate, and detects optimal exercise intensity on the basis of a bending curve indicating exercise intensity.

Furthermore, a double product of amplitude of first heart sound and a heart rate has a high correlation with secretion quantity of adrenalin. This is considered because a double product of amplitude of first heart sound and a heart rate has a high correlation with oxygen consumption in a heart, and hence, reflects increasing neurotransmitter which induces secretion of adrenalin.

For instance, FIG. 8 illustrates a graph having an axis of ordinates (Y-axis) indicative of both a double product and a secretion quantity of adrenalin (blood concentration) and an axis of abscissas (X-axis) indicative of an exercise intensity in such a condition that grade exercise intensity is gradually increased by causing a target person to perform grade exercise from a condition in which he/she is resting to a condition in which heavy load acts on him/her. A secretion quantity of adrenalin was measured by a conventional process, that is, by taking blood from a target person who is exercising. In light of the graph of FIG. 8, it is understood that a double product of amplitude of first heart sound and a heart rate has a correlation with a secretion quantity of adrenalin. It is further understood that the approximation line L5 teaches that a bending point P at which a gradient of the approximation line L remarkably increases indicates optimal exercise intensity, and that the bending point P defines an intensity threshold at which a secretion quantity of adrenalin starts to increase significantly.

Accordingly, it was difficult in a conventional process to measure a secretion quantity of adrenalin while a target person is exercising, because a secretion quantity of adrenalin was measured by taking blood of a target person each time he/she was tested, it is now possible to detect optimal exercise intensity through the use of a double product of an amplitude of first heart sound and a heart rate, and further possible to readily and accurately measure an intensity threshold at which a secretion quantity of adrenalin starts to increase significantly.

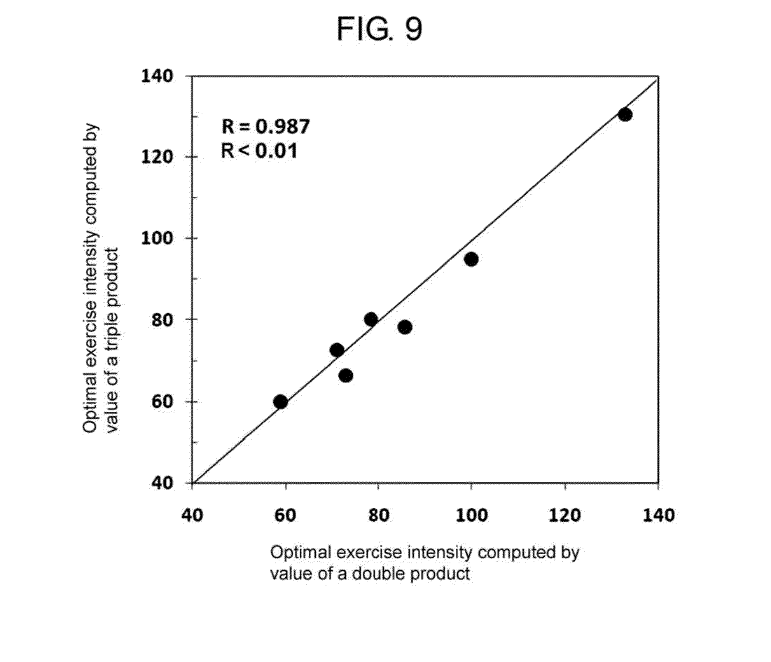

The apparatus 1 for detecting somatic data in accordance with the first embodiment measures optimal exercise intensity by value of a double product. A triple product of amplitude of first heart sound, a heart rate, and second heart sound may be employed for measuring optimal exercise intensity. As illustrated in FIG. 9, it was found that a correlation between a double product and a triple product is almost equal to one. Consequently, in the apparatus 1 for detecting somatic data, the exercise intensity computing means 510 detects optimal exercise intensity by value of a double product without computing a triple product.

On detection of an optimal exercise intensity, the exercise intensity computing means 510 stores the detected optimal exercise intensity into the storage means 517 together with predetermined identification data (for instance, a name or an ID number) identifying a target person. By storing the optimal exercise intensity into the storage means 517 in association with identification data, the apparatus 1 for detecting somatic data can record optimal exercise intensity in association with each target person.

Since the graph of FIG. 7 can be illustrated during steps carried out by the exercise intensity computing means 510 for detecting an optimal exercise intensity, the graph may be displayed in the display means 6 through the display controlling means 514, or printed onto paper medium by means of the print means 7 through the printing controlling means 515.

By detecting an optimal exercise intensity in the above-mentioned way, since the annunciating means 513 makes annunciation when grade exercise is equal to or greater than an optimal exercise intensity, or when grade exercise exceeds an optimal exercise intensity and then becomes equal to or greater than a predetermined exercise intensity, it is possible for a target person to avoid exercising at a harmful load.

Furthermore, if optimal exercise intensity for a certain target person was recorded, the annunciating means 513 reads an optimal exercise intensity associated with identification data of the certain target person, out of the storage means 517, compares the optimal exercise intensity with exercise intensity data received from the exercise machine A to thereby make annunciation. Thus, when the certain target person performs grade exercise again, he/she can perform optimal grade exercise without measuring optimal exercise intensity again.

It is explained herein below how the aerobic exercise capacity detecting means 516 detects a maximum volume of oxygen taken by a target person.

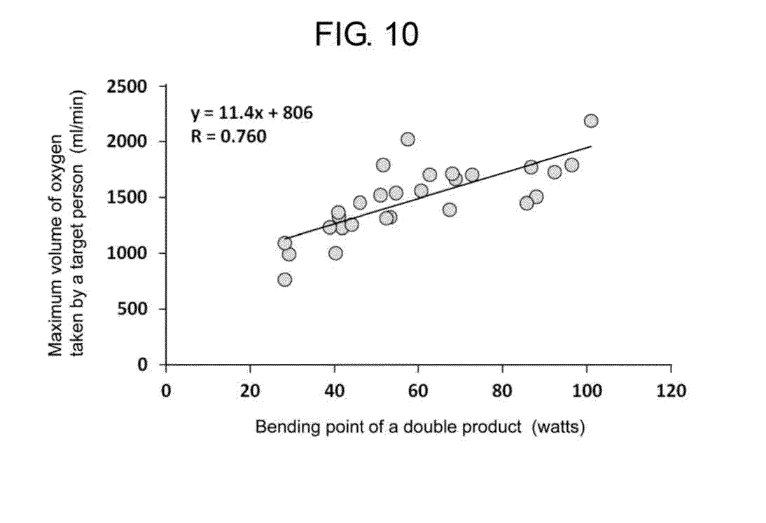

The graph illustrated in FIG. 10 has an x-axis indicative of optimal exercise intensities (bending points of double products) of a plurality of target persons, and a y-axis indicative of a maximum volume of oxygen taken by a target person.

As is understood in light of the graph of FIG. 10, the approximation line (regression line) indicating a relation between optimal exercise intensity and maximum volume of oxygen taken by a target person can be expressed by the following relational expression (8).

y=11.4x+806 (8)

Estimating the relational expression (8) with Pearson's correlation coefficients, since the correlation coefficient R is equal to 0.760 (significance level P<0.01), it is understood that the relational expression (8) indicates a high correlation.

The aerobic exercise capacity detecting means 516 can calculate a maximum volume of oxygen taken by a target person by putting optimal exercise intensity detected by the exercise intensity computing means 510, into the relational expression (8), that is, the expression for calculating aerobic exercise capacity.

As mentioned above, the exercise intensity computing means 510 computes the double product data of a target person, and hence, it is possible to measure optimal exercise intensity. The optimal exercise intensity being measured, the aerobic exercise capacity detecting means 516 can compute aerobic exercise capacity of the target person, and then, it is possible to compute a maximum volume of oxygen taken by the target person on the basis of the computed aerobic exercise capacity.

### Second Embodiment

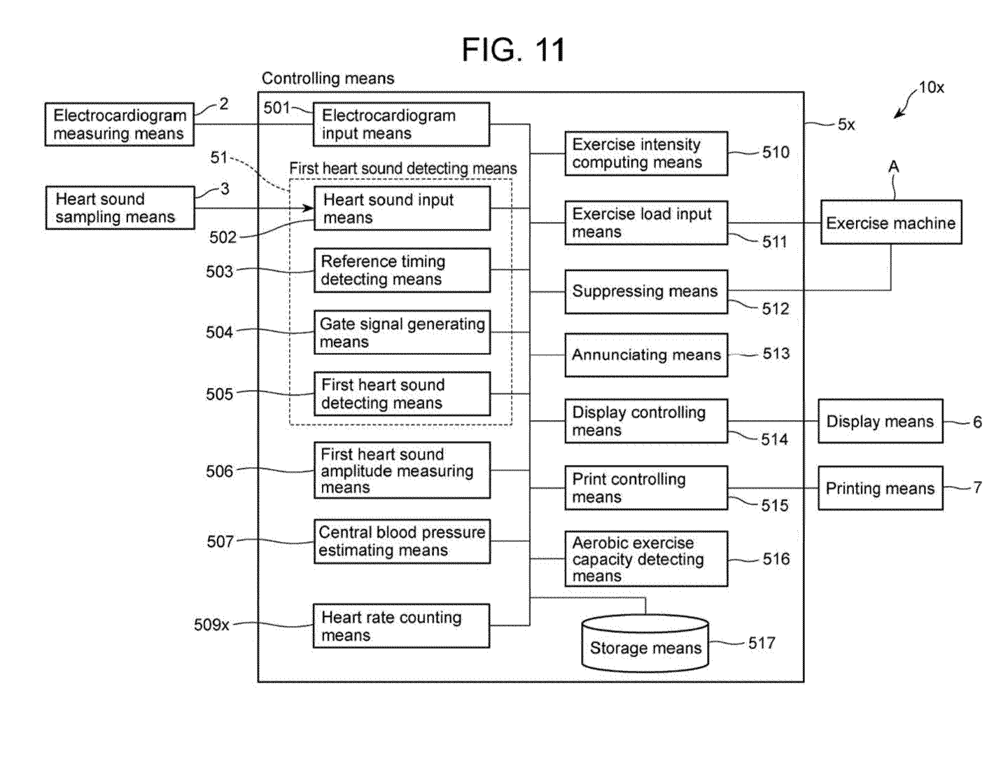

The apparatus 10x for detecting somatic data in accordance with the second embodiment of the present invention is characterized by the heart rate computed on the basis of R-wave or first heart sound. In FIG. 11, parts corresponding to those illustrated in FIG. 2 have been provided with the same reference numerals, and are not explained.

The heart rate counting means 509x equipped in the controlling means 5x of the apparatus 10x illustrated in FIG. 11 counts a heart rate by measuring a period P1 (see FIG. 5) between R-wave detected by the reference timing detecting means 503 and a subsequent R-wave.

Furthermore, the heart rate counting means 509x is able to count a heart rate on the basis of the period P1 in accordance with the first heart sound S1 detected by the first heart sound detecting means 505.

As mentioned above, it is possible to count a heart rate on the basis of a period between R-waves or first heart sounds both measured during steps carried out for measuring an exercise intensity, without particularly providing means (the heart rate measuring means 4) for counting a heart rate unlike the apparatus 1 for detecting somatic data in accordance with the first embodiment.

### INDUSTRIAL APPLICABILITY

The present invention is suitable for measuring central blood pressure which is a blood pressure of a region from which aorta extends, and a stress acting on heart, and suitable in particular for measuring an optimal exercise intensity.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

The entire disclosure of Japanese Patent Application No. 2010-229873 filed on Oct. 12, 2010 including specification, claims, drawings and summary is incorporated herein by reference in its entirety.

1-7. (canceled)

8. An apparatus for detecting somatic data, comprising:
first means for sampling heart sound of a target person while said target person is exercising;
second means for detecting first heart sound on the basis of said heart sound;
third means for measuring an amplitude of said first heart;
fourth means for counting a heart rate of said target person while said target person is exercising;
fifth means for storing a heart rate of said target person to be measured when said target person is resting, and an amplitude of first heart sound of said target person to be measured when said target person is resting; and
sixth means for computing a double product of a ratio between said heart rate of said target person to be measured when said target person is resting and said heart rate to be measured when said target person exercises, and a ratio between said amplitude of first heart sound of said target person to be measured when said target person is resting and said amplitude of first heart sound of said target person to be measured when said target person exercises, and detecting an optimal exercise intensity of said target person on the basis of said double product.

9. The apparatus as set forth in claim 8, wherein said sixth means detects an exercise intensity at which a line approximate to a distribution of said double product is bending, as optimal exercise intensity.

10. The apparatus as set forth in claim 9, wherein said sixth means
divides said double product into a first group covering said double product before a bending point appears and a second group covering said double product after a bending point appeared,
computes a regression line of said first group as a first approximation straight line,
computes a regression line of said second group as a second approximation straight line,
selects, among combinations of said first approximation straight line and said second approximation straight line, a combination which minimize a sum of a residual sum of squares of said first approximation straight line and a residual sum of squares of said second approximation straight line, and
detects an intersection point of said first and second approximation straight lines of the selected combination as said optimal exercise intensity.

11. The apparatus as set forth in claim 8, further seventh means which puts said optimal exercise intensity detected by said sixth means into a relational expression derived from a correlation between said optimal exercise intensity and a maximum volume of oxygen taken by a target person during grade exercise, obtained by measuring a plurality of target persons, to thereby detect a maximum volume of oxygen taken by said target person during grade exercise.

12. The apparatus as set forth in claim 8, wherein said fifth means stores a central blood pressure to be measured when said target person is resting,
said apparatus further includes eighth means for computing central blood pressure while said target person is being tested on the basis of central blood pressure to be measured while said target person is resting, in accordance with a ratio between said amplitude of first heart sound of said target person to be measured while said target person is resting as standard data and received from said first heart sound amplitude measuring means, and said amplitude of first heart sound of said target person to be measured while said target person is being tested.

13. The apparatus as set forth in claim 12, wherein said eighth means estimates a central blood pressure of said target person while said target person is being tested, in accordance with a relational expression between central blood pressure and an amplitude of said first heart sound, said relational expression being defined for each of target persons on the basis of a central blood pressure and an amplitude of first heart sound of a target person both to be measured while said target person is resting, and a central blood pressure and an amplitude of first heart sound of a target person both to be measured while said target person is exercising.

14. The apparatus as set forth in claim 8, wherein said second means includes:
second-A means for measuring electrocardiogram of said target person;
second-B means for detecting an R-wave out of said electrocardiogram;
second-C means for generating a gate signal, in accordance with timing at which said R-wave is generated, indicative of a certain period including first heart sound corresponding to said R-wave; and
second-D means for detecting first heart sound on the basis of said heart sound taken while said gate signal is being generated.

15. A method of detecting somatic data, comprising:
first step of sampling heart sound of a target person to be measured while said target person is exercising;
second step of detecting first heart sound on the basis of said heart;
third step of measuring an amplitude of said first heart;
fourth step of counting a heart rate of said target person to be measured while said target person is exercising;
fifth step of storing a heart rate of said target person to be measured when said target person is resting, and an amplitude of first heart sound of said target person to be measured when said target person is resting; and
sixth step of computing a double product of a ratio between said heart rate of said target person to be measured when said target person is resting and said heart rate to be measured when said target person exercises, and a ratio between said amplitude of first heart sound of said target person to be measured when said target person is resting and said amplitude of first heart sound of said target person to be measured when said target person exercises, and detecting an optimal exercise intensity of said target person on the basis of said double product.

16. The method as set forth in claim 15, wherein said sixth step includes detecting an exercise intensity at which a line approximate to a distribution of said double product is bending, as optimal exercise intensity.

17. The method as set forth in claim 15, wherein said sixth step includes:
dividing said double product into a first group covering said double product before a bending point appears and a second group covering said double product after a bending point appeared;
computing a regression line of said first group as a first approximation line;
computing a regression line of said second group as a second approximation line;
selecting, among combinations of said first approximation line and said second approximation line, a combination which minimize a sum of a residual sum of squares of said first approximation line and a residual sum of squares of said second approximation line; and
detecting an intersection point of said first and second approximation straight line of the selected combination as said optimal exercise intensity.

18. The method as set forth in claim 15, further including seventh step of putting said optimal exercise intensity detected in said sixth step into a relational expression derived from a correlation between said optimal exercise intensity and a maximum volume of oxygen taken by a target person during grade exercise, obtained by measuring a plurality of target persons, to thereby detect a maximum volume of oxygen taken by said target person during grade exercise.

19. The method as set forth in claim 15, wherein a central blood pressure to be measured when said target person is resting, is stored in said fifth step,
said method further includes eighth step of computing central blood pressure while said target person is being tested on the basis of central blood pressure to be measured while said target person is resting, in accordance with a ratio between said amplitude of first heart sound of said target person to be measured while said target person is resting as standard data and received from said first heart sound amplitude measuring means, and said amplitude of first heart sound of said target person to be measured while said target person is being tested.

20. The method as set forth in claim 19, wherein said eighth step includes estimating central blood pressure of said target person while said target person is being tested, in accordance with a relational expression between central blood pressure and an amplitude of said first heart sound, said relational expression being defined for each of target persons on the basis of central blood pressure and an amplitude of first heart sound of a target person both to be measured while said target person is resting, and central blood pressure and an amplitude of first heart sound of a target person both to be measured while said target person is exercising.

21. The method as set forth in claim 15, wherein said second step includes:
measuring electrocardiogram of said target person;
detecting an R-wave out of said electrocardiogram;
generating a gate signal, in accordance with a timing at which said R-wave is generated, indicative of a certain period including first heart sound corresponding to said R-wave; and
detecting first heart sound on the basis of said heart sound taken while said gate signal is being generated.

22. A computer-readable storage medium containing a set of instructions for causing a computer to carry out a method of detecting somatic data, the set of instructions comprising:
first instruction for detecting first heart sound on the basis of heart sound of a target person to be measured while said target person is exercising;
second instruction for measuring an amplitude of said first heart sound;
third instruction for storing a heart rate of said target person to be measured when said target person is resting, and an amplitude of first heart sound of said target person to be measured when said target person is resting; and
fourth instruction for computing a double product of a ratio between said heart rate of said target person to be measured when said target person is resting and said heart rate to be measured when said target person exercises, and a ratio between said amplitude of first heart sound of said target person to be measured when said target person is resting and said amplitude of first heart sound of said target person to be measured when said target person exercises, and detecting an optimal exercise intensity of said target person on the basis of said double product.

23. The computer-readable storage medium as set forth in claim 15, wherein said sixth step includes detecting an exercise intensity at which a line approximate to a distribution of said double product is bending, as optimal exercise intensity.

24. The computer-readable storage medium as set forth in claim 23, wherein said fourth instruction includes:
dividing said double product into a first group covering said double product before a bending point appears and a second group covering said double product after a bending point appeared;
computing a regression line of said first group as a first approximation line;
computing a regression line of said second group as a second approximation line;
selecting, among combinations of said first approximation line and said second approximation line, a combination which minimize a sum of a residual sum of squares of said first approximation line and a residual sum of squares of said second approximation line; and
detecting an intersection point of said first and second approximation straight line of the selected combination as said optimal exercise intensity.

25. The computer-readable storage medium as set forth in claim 22, wherein said instructions further includes fifth instruction for putting said optimal exercise intensity detected by said fourth instruction into a relational expression derived from a correlation between said optimal exercise intensity and a maximum volume of oxygen taken by a target person during grade exercise, obtained by measuring a plurality of target persons, to thereby detect a maximum volume of oxygen taken by said target person during grade exercise.

26. The computer-readable storage medium as set forth in claim 22, wherein a central blood pressure to be measured when said target person is resting is stored in said third instruction,
said instructions further includes sixth instruction for computing a central blood pressure while said target person is being tested on the basis of central blood pressure to be measured while said target person is resting, in accordance with a ratio between said amplitude of first heart sound of said target person to be measured while said target person is resting as standard data and received from said first heart sound amplitude measuring means, and said amplitude of first heart sound of said target person to be measured while said target person is being tested.

27. The computer-readable storage medium as set forth in claim 26, wherein said sixth instruction includes estimating central blood pressure of said target person while said target person is being tested, in accordance with a relational expression between central blood pressure and amplitude of said first heart sound, said relational expression being defined for each of target persons on the basis of central blood pressure and amplitude of first heart sound of a target person both to be measured while said target person is resting and central blood pressure and amplitude of first heart sound of a target person both to be measured while said target person is exercising.

28. The computer-readable storage medium as set forth in claim 22, wherein said first instruction includes:
measuring electrocardiogram of said target person;
detecting an R-wave out of said electrocardiogram;
generating a gate signal, in accordance with a timing at which said R-wave is generated, indicative of a certain period including first heart sound corresponding to said R-wave; and
detecting first heart sound on the basis of said heart sound taken while said gate signal is being generated.
